(19) United States
(12) Patent Application Publication
(10) Pub. No.: US 2023/0395203 A1
(43) Pub. Date: Dec. 7, 2023

(54) COMPUTER-IMPLEMENTED SYSTEM AND METHOD FOR PROVIDING OPTIMIZED PROCESS IN CLINICAL RESEARCH

(71) Applicant: NUSRAT AHMED, Burr Ridge, IL (US)

(72) Inventor: NUSRAT AHMED, Burr Ridge, IL (US)

(21) Appl. No.: 17/833,922

(22) Filed: Jun. 7, 2022

(51) Int. Cl. G16H 10/20; G16H 40/20; G16H 10/60

(52) U.S. Cl. CPC G16H 10/20; G16H 40/20; G16H 10/60

(57) ABSTRACT

A method for providing an optimized process in clinical research, comprising enabling research participant, site manager to input research participants' information by enrolment and retention module on research participant computing device, research site manager computing device. Identifying research participants' information stored in database and generating list of qualified research participants by list generating module. Enabling qualified research participants to schedule appointment with site manager by appointment scheduling module and assigning principal investigators to screen research participants. Enabling principal investigators to input screening data by primary source data capture module after screening research participants in real-time. Collecting screening data to calculate accounts receivable from sponsor by financial module and sending screening data to data review and reports generating module. Generating screening reports by data review and reports generating module and sending screening reports to invoice generating module thereby generating invoice. Sending invoice to sponsor computing device and enabling sponsor to review invoice.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to a system and computer-implemented method for providing an optimized process in clinical research. More particularly, the present disclosure relates to a system and method for collecting, sorting and processing research participants' data to provide an optimized process in clinical research.

### BACKGROUND

Clinical research determines the effectiveness and safety of new drugs, treatments, devices, and diagnostic products. Traditionally, healthcare providers store patients' information in paper filing systems. The manual, paper-based practice of collecting patients' information requires a significant amount of the healthcare provider's time. The manual, paper-based method of managing patient information is inefficient, labour-intensive, and requires many checks and balances to ensure accurate processing of patient information. The problem with the existing systems is collecting, sorting, and processing huge amounts of patient information to manage clinical research effectively.

Further, ensuring the safety and quality of the patients' information is paramount, and flaws in the management process threaten the safety and quality of the patients' information. The current systems available to assist with the safety and quality of the patients' information are cumbersome, inefficient, and expensive. Hence, a system needs to provide an optimized process that efficiently manages clinical research.

In the light of the aforementioned discussion, there exists a need for a certain system with novel methodologies that would overcome the above-mentioned challenges.

### SUMMARY

The following invention presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An objective of the present disclosure is directed towards a system and method for providing an optimized process in clinical research.

Another objective of the present disclosure is directed towards the system that improves efficiency and quality in clinical research.

Another objective of the present disclosure is directed towards the system efficiently managing the clinical research.

Another objective of the present disclosure is directed towards the system optimizes the process for the research participant (subject), research sites, and sponsors.

Another objective of the present disclosure is directed towards the system used for clinical research, clinical trials, clinical applications, or any other variations.

Another objective of the present disclosure is directed towards the system used for the testing of drugs, medical treatments, diagnosis tools, and medical techniques.

Another objective of the present disclosure is directed towards the system used for human trials, primate trials, and rodent trials.

Another objective of the present disclosure is directed towards optimizing the data collection and data delivery process resulting in faster and more accurate data collection which will result in bringing new therapies and treatments to market faster.

Another objective of the present disclosure is directed towards storing pre-screened and consented research participants' medical information in the central database and enabling the research site manager and the sponsor to easily access the research studies/clinical research.

Another objective of the present disclosure is directed towards capturing the research participants' information in the primary source data capture module when the research participant signs in and arrives for their research study visit at the research site location.

Another objective of the present disclosure is directed towards enabling the sponsor to review the research participants' information captured in real-time by the research site manager thereby eliminating and saving time in travel, scanning, and faxing paper charts.

Another objective of the present disclosure is directed towards enabling the research site manager and the principal investigator to review the research participants' information and generate screening reports as to the amount of money that is owed to the site.

Another objective of the present disclosure is directed towards generating an invoice simultaneously and periodically thereby sending it to the sponsor directly.

Another objective of the present disclosure is directed towards providing detailed metrics of the principal investigator and the research site manager to exactly represent how many studies were completed by the physicians, in which therapeutic area, with real-time enrollment numbers, quality, and audit information.

In an embodiment of the present disclosure, a system comprising a research participant's computing device, a research site manager's computing device, and a principal investigator's computing device, and a sponsor's computing device comprises a processor and a memory.

In another embodiment of the present disclosure, the memory configured to store a research process optimizing module configured to collect, sort and process one or more research participants' information to provide the optimized process in the clinical research.

In another embodiment of the present disclosure, the research process optimizing module comprising an enrolment and retention module configured to enable at least of: a research participant; and a site manager to input the one or more research participants' information on at least one of: the research participant's computing device; and the research site manager computing device over a network.

In another embodiment of the present disclosure, a list generating module configured to identify the one or more research participants' information stored in the central database and generate a list of qualified research participants to participate in the clinical research based on at least one of: an exclusion criteria; and an inclusion criteria; and the one or more research participants' information by accelerating Ai (artificial intelligence) implementation at an enterprise level.

In another embodiment of the present disclosure, an appointment scheduling module configured to enable the list of qualified research participants to input additional patient's information on the research participant's computing device and to schedule an appointment with the site manager for research study screening.

In another embodiment of the present disclosure, the appointment scheduling module configured to enable the list of qualified research participants to send a request from the research participant's computing device to the research site manager's computing device for scheduling the appointment.

In another embodiment of the present disclosure, the appointment scheduling module configured to assign one or more principal investigators for screening one or more research participants based on the one or more research participants' information.

In another embodiment of the present disclosure, an primary source data capturing module configured to enable one or more principal investigators to input screening data of the one or more research participants on the principal investigator's computing device after screening the one or more research participants at a research site location in real-time.

In another embodiment of the present disclosure, a financial module configured to collect the screening data in real-time and calculate accounts receivable from a sponsor thereby send the screening data to a data review and reports generating module.

In another embodiment of the present disclosure, the data review and reports generating module configured to generate one or more screening reports as the amount of money that is owed to a research site location and send the one or more screening reports to an invoice generating module.

In another embodiment of the present disclosure, an invoice generating module configured to generate an invoice based on the one or more screening reports of the one or more research participants and send the invoice to the sponsor's computing device.

In another embodiment of the present disclosure the data review and reports generating module configured to enable the sponsor to review the invoice on the sponsor computing device.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 is a block diagram depicting a schematic representation of a system for an optimized process in clinical research, in accordance with one or more exemplary embodiments.

FIG. 2 is a block diagram depicting an embodiment of the research process optimizing module 110 shown in FIG. 1, in accordance with one or more exemplary embodiments.

FIG. 3 is a flow diagram depicting a method for providing an optimized process in clinical research, in accordance with one or more exemplary embodiments.

FIG. 4 is a flow diagram depicting a method of an optimized process that improves efficiency and quality in clinical research, in accordance with one or more exemplary embodiments.

FIG. 5 is another exemplary flow diagram depicting a method of an optimized process that improves efficiency and quality in clinical research, in accordance with one or more exemplary embodiments.

FIG. 6 is a flow diagram depicting a method for sorting the research participants' information into a central database and selecting the suitable research participants thereby contacting the suitable research participants for the relevant trial in clinical research, in accordance with one or more exemplary embodiments.

FIG. 7 is a flow diagram depicting a method for scheduling an appointment and screening the research participants, and entering the relevant research participants' information into the primary source data capturing module thereby generating the financial information and invoices automatically, in accordance with one or more exemplary embodiments.

FIG. 8A is a flow diagram depicting a method for uploading documents by the e-regulatory module, in accordance with one or more exemplary embodiments.

FIG. 8B is a flow diagram depicting a method for sharing the research participant information between the research participant, the research site, and the sponsor, in accordance with one or more exemplary embodiments.

FIG. 9A is another exemplary flow diagram depicting the method for uploading documents by the e-regulatory module, in accordance with one or more exemplary embodiments.

FIG. 9B is another exemplary flow diagram depicting the method for sharing the research participant information between the research participant, the research site, and the sponsor, in accordance with one or more exemplary embodiments.

FIG. 10 is a block diagram illustrating the details of a digital processing system in which various aspects of the present disclosure are operative by the execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

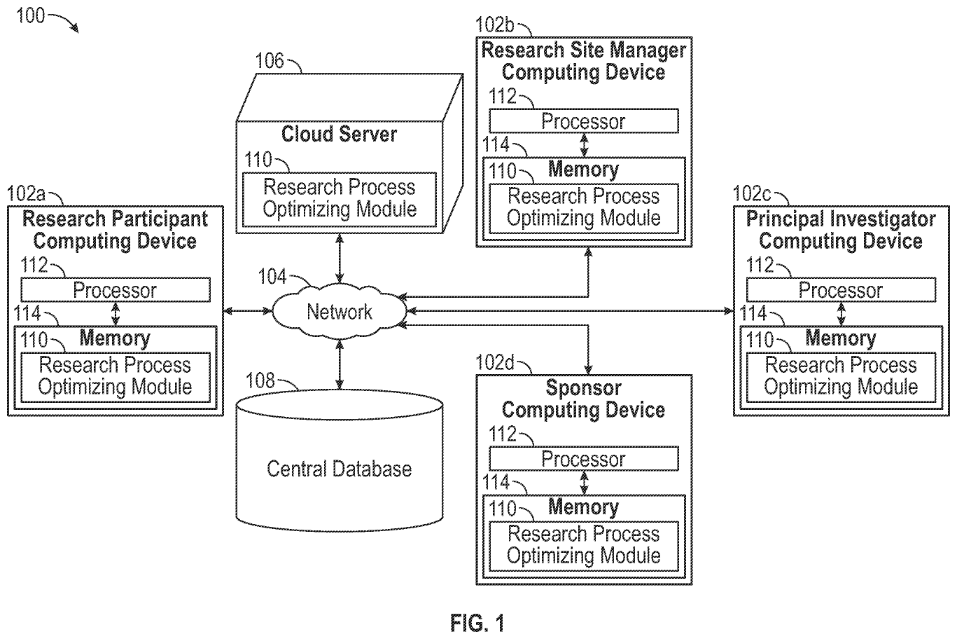

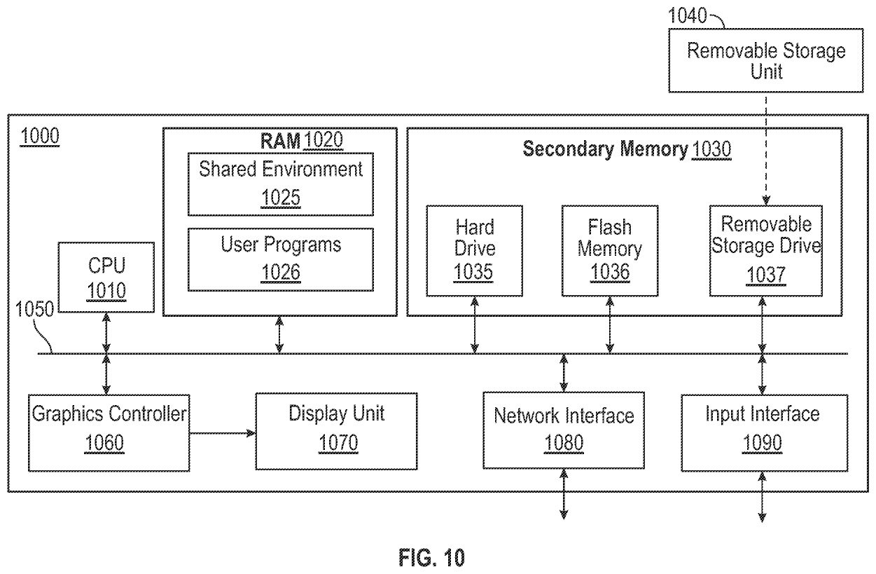

Referring to FIG. 1 is a block diagram 100 depicting a schematic representation of a system for an optimized process in clinical research, in accordance with one or more exemplary embodiments. The system 100 includes a research participant computing device 102a, a research site manager computing device 102b, a principal investigator computing device 102c, and a sponsor computing device 102d, a network 104, a cloud server 106, a central database 108, a processor 112 and a memory 114. The research participant computing device 102a, the research site manager computing device 102b, the principal investigator computing device 102c, and the sponsor computing device 102d may be connected to one or more computing devices via the network 104. The research participant computing device 102a, the research site manager computing device 102b, the principal investigator computing device 102c, and the sponsor computing device 102d and the cloud server 106 includes the memory 114 configured to store a research process optimizing module 110. The processor 112 may be a central processing unit and/or a graphics processing unit (As shown in FIG. 10).

The network 104 may include, but not limited to, an Internet of things (IoT network devices), an Ethernet, a wireless local area network (WLAN), or a wide area network (WAN), a Bluetooth low energy network, a ZigBee network, a WIFI communication network e.g., the wireless high-speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure. The network 104 may be configured to provide access to different types of users. The research participant computing device 102a, the research site manager computing device 102b, the principal investigator computing device 102c, and the sponsor computing device 102d may include, but is not limited to, a personal digital assistant, smartphones, personal computers, a mobile station, computing tablets, a handheld device, an internet-enabled calling device, an internet-enabled calling software, a telephone, a mobile phone, a digital processing system, and so forth. The research process optimizing module 110 is accessed as a mobile application, web application, software that offers the functionality of accessing mobile applications, and viewing/processing of interactive pages.

Although the research participant computing device 102a, the research site manager computing device 102b, the principal investigator computing device 102c, and the sponsor computing device 102d are shown in FIG. 1, an embodiment of the system 100 may support any number of computing devices. The research participant computing device 102a may be operated by a research participant. The research participant may include but not limited to, a subject, a client, a patient, a regulatory body, and so forth. The research site manager computing device 102b may be operated by a research site manager. The research site manager may include but not limited to, the site manager, research site staff, site location staff, site members, therapeutic area staff, and so forth. The principal investigator computing device 102c may be operated by a principal investigator. The principal investigator may include, but not limited to, a medical doctor, a physician, a surgeon, a consultant, and so forth. The sponsor computing device 102d may be operated by a sponsor. The sponsor may include, but not limited to, a supporter, a promoter, an agent, and so forth. the research participant computing device 102a, the research site manager computing device 102b, the principal investigator computing device 102c, and the sponsor computing device 102d supported by the system 100 is realized as a computer-implemented or computer-based device having the hardware or firmware, software, and/or processing logic needed to carry out the computer-implemented methodologies described in more detail herein.

In accordance with one or more exemplary embodiments of the present disclosure, the research process optimizing module 110 may be configured to enable the research participant to sign-up by providing the research participant identity credentials on the research participant computing device 102a. The research participant identity credentials may include, but not limited to, name, mobile number, age, research gender, occupation, email identity, password, and so forth. The research process optimizing module 110 may be configured to enable the research participant to input the research participant information on the research participant computing device 102a. The research process optimizing module 110 may be configured to enable the research site manager to sign-up by providing the research site manager identity credentials on the research site manager computing device 102b. The research site manager's identity credentials may include, but not limited to, name, mobile number, age, gender, occupation, email identity, password, and so forth. The research process optimizing module 110 may also be configured to enable the research site manager to input the research participant information on the research site manager computing device 102a. The research participant information may include, but not limited to, diagnosis and medical history, patient's demographic information (e.g., age, weight, gender, race, income, and geographic location) and health-related information (e.g., clinician documentation of observations, thoughts and actions, treatments administered, patient history, medication and allergy lists, vaccine administration lists, laboratory reports, X-rays, charts, progress notes, consultation reports, procedure notes, hospital reports, correspondence, and test results) and so forth.

The research process optimizing module 110 may be configured to enable the principal investigator to sign-up by providing the required principal investigator identity credentials on the principal investigator computing device 102c. The principal investigator's identity credentials may include, but not limited to, name, mobile number, age, gender, occupation, email identity, password, and so forth. The principal investigator may include, but not limited to, a medical doctor, a physician, a surgeon, a consultant and so forth. The research process optimizing module 110 may be configured to enable the sponsor to sign-up by providing the sponsor identity credentials on the sponsor computing device 102d. The sponsor identity credentials may include, but not limited to, name, mobile number, age, gender, occupation, email identity, password, and so forth. The research process optimizing module 110 may be configured to enable the sponsor to review the invoice on the sponsor computing device 102d. The sponsor may include, but not limited to, a supporter, a promoter, an agent, and so forth.

The research process optimizing module 110 may be configured to update the research participant information in the central database 108 over the network 104. The central database 108 may be configured to store the research participant information and enables the principal investigator, the research site manager and the sponsor to access the central database 108 by the research process optimizing module 110 on the principal investigator computing device 102c, the research site manager computing device 102b, and the sponsor computing device 102d over the network 104.

The central database 108 may be configured to store algorithms, artificial intelligence, and machine learning techniques and generates a list of qualified subjects (list of research participants) based on the inclusion/exclusion criteria of the research participants' information. The inclusion/exclusion criteria may include, who can participate in the research study and who cannot. The research process optimizing module 110 may be configured to send a notification to the group of qualified research participants to seek interest in the research study. The notification may include, but not limited to, a text message, a SMS, a voice note, and so forth.

The patient's details and the medical records are stored in custom defined tables enabling the software to produce patient details based on diagnosis code and medical history. Each clinical trial is designed with specific criteria, therefore, making it extremely difficult to determine which patients qualify for which clinical trial. The list of qualified patients is determined using algorithms and custom-built modules. The extracted list may save time and effort and enable access to patients to participate in clinical trials. Therefore bringing new drugs and therapies to market faster for the greater good of humanity.

The research process optimizing module 110 may be configured to enable the group of qualified research participants to request the research site manager for a research study screening appointment, and/or to add the additional information, or schedule an online appointment. The research site manager computing device 102b may be configured to receive the request from the research participant computing device 102a to schedule the appointment for the group of qualified research participants. The research process optimizing module 110 on the research site manager computing device 102b may be configured to send a reminder text to the research participant computing device 102a based on the scheduled appointment.

In accordance with one or more exemplary embodiments of the present disclosure, the qualified group of research participants may visit a research site location for the research study screening appointment. The qualified group of research participants may be screened by the principal investigators for the research study at the research site location. Further, the research process optimizing module 110 may be configured to capture the screening data of the group of research participants on the research site manager computing device 102b. The screening data may include, but not limited to, labs and study visit procedures of the qualified group of research participants, and so forth. The study visit procedures may be varied and may require different procedures to perform which are highly customized depending on the study-specific needs. The research process optimizing module 110 may be configured to enable the principal investigator to sign off on the principal investigator computing device 102c in real-time while performing the history and medical examination of the qualified group of research participants.

The research process optimizing module 110 on the sponsor computing device 102d may be configured to enable the sponsor to review the screening data captured in real-time at the research site location. The research process optimizing module 110 may be configured to eliminate and save time in travelling, scanning, and faxing paper charts.

In accordance with one or more exemplary embodiments of the present disclosure, the research process optimizing module 110 may be configured to enable the research site manager to review the screening data and generates screening reports as the amount of money that is owed to the research site location. The research process optimizing module 110 may be configured to generate an invoice simultaneously and periodically based on the screening reports and send the invoice to the sponsor computing device 102d over the network 104. The research process optimizing module 110 may be configured to update the invoice of the amount receivable from the sponsor.

In accordance with one or more exemplary embodiments of the present disclosure, the research process optimizing module 110 may be configured to enable the principal investigator and the sponsor to update the principal investigator metrics and the site metrics on the principal investigator computing device 102c and the sponsor computing device 102d in real-time. The research process optimizing module 110 may be configured to provide detailed metrics for the principal investigator and the sponsor to exactly represent how many studies were completed by the physicians, in which therapeutic area, with real-time enrollment numbers, quality, and audit information. The research process optimizing module 110 may be configured to store research sites capabilities with pictures and the patient testimonials and are made available for the sponsor to review on the sponsor computing device 102d. The research process optimizing module 110 may be configured to enable the research site manager to provide CRA (Clinical Research Associate) reviews to the sponsor in real-time.

In accordance with one or more exemplary embodiments of the present disclosure, the research process optimizing module 110 may be configured to provide access to the research site manager to upload the documents such as econsent, patients' lab reports, and the like. which can be sent to each specific individual (each qualified research participant) for electronic signatures, and with a few clicks, submitted to the sponsor for review and then to the institutional review board (IRB) for approval. The research process optimizing module 110 may be configured to enable the research site manager to provide feedback to the sponsor. The research process optimizing module 110 may be configured to enable the sponsor to provide feedback to the research site manager.

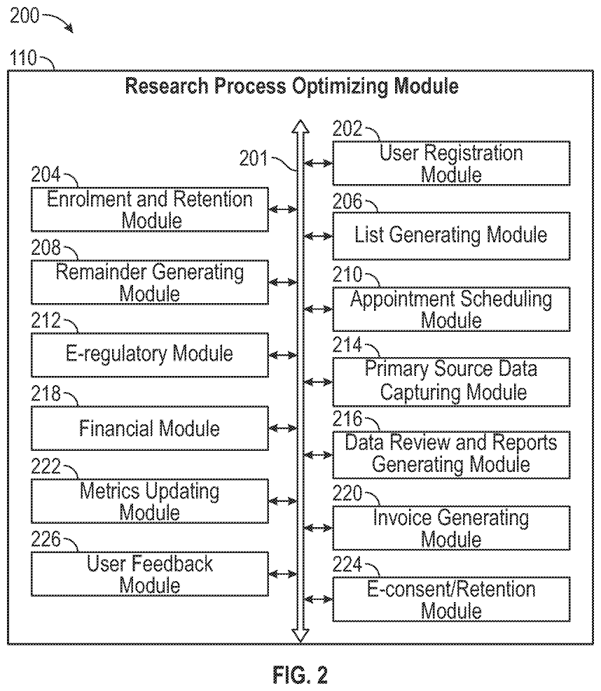

Referring to FIG. 2 is a block diagram 200 depicting an embodiment of the research process optimizing module 110 shown in FIG. 1, in accordance with one or more exemplary embodiments. The research process optimizing module 110 includes a bus 201, a user registration module 202, an enrollment and retention module 204, a list generating module 206, text remainder generating module 208, an appointment scheduling module 210, an e-regulatory module 212, an primary source data capturing module 214, data review and reports generating module 216, a financial module 218, an invoice generating module 220, a principal investigator/site metrics updating module 222, e-consent/retention module 224, and a user feedback module 226.

The bus 201 may include a path that permits communication among the modules of the research process optimizing module 110 installed on the research participant computing device 102a, the research site manager computing device 102b, the principal investigator computing device 102c, the sponsor computing device 102d, and the cloud server 106. The term “module” is used broadly herein and refers generally to a program resident in the memory of the research participant computing device 102a, the research site manager computing device 102b, the principal investigator computing device 102c, the sponsor computing device 102d, and the cloud server 106.

The user registration module 202 may be configured to enable the research participant to register and sign-up by providing the research participant identity credentials on the research participant computing device 102a. The research participant identity credentials may include, but not limited to, research participant name, research participant mobile number, age, gender, occupation, email identity, password, and so forth. The research participant may include but not limited to, a research participant, a subject, a client, a patient, a regulatory body, and so forth.

The user registration module 202 may also be configured to enable the research site manager to register and sign-up by providing the research site manager identity credentials on the research site manager computing device 102b. The research site manager identity credentials may include, but not limited to, research site manager name, research site manager mobile number, age, gender, occupation, email identity, password, and so forth. The research site manager may include but not limited to, site manager, research site staff, site location staff, site members and so forth. The user registration module 202 may also be configured to enable the principal investigator to register and sign-up by providing the principal investigator identity credentials on the principal investigator computing device 102c. The principal investigator may include, but not limited to, a principal investigator, a physician, a medical doctor, a surgeon and so forth. The user registration module 202 may be configured to enable the sponsor to register and sign-up by providing the sponsor identity credentials. The sponsor identity credentials may include, but not limited to, sponsor name, sponsor mobile number, age, gender, occupation, email identity, password, and so forth. The sponsor may include, but not limited to, a sponsor, a supporter, a promoter, an agent, and so forth.

The enrollment and retention module 204 may be configured to optimize and improve the research participant experience and engagement in the clinical research. The enrollment and retention module 204 may be configured to enable the quality patients (research participants) to enroll in clinical trials. The enrollment and retention module 204 configured to enhance the research participant experience and improve research participant retention in short term and especially in long-term clinical trials. The enrollment and retention module 204 may be configured to enable the research participant to input the research participant information on the research participant computing device 102a. The enrollment and retention module 204 may be configured to enable the research site manager to sign-up by providing the research site manager identity credentials on the research site manager computing device 102b. The enrollment and retention module 204 may also be configured to enable the research site manager to input the research participant information on the research site manager computing device 102b. The research participant information/the patient information may include, but not limited to, diagnosis and medical history, patients' demographic information (e.g., age, weight, gender, race, income, and geographic location) and health-related information (e.g., clinician documentation of observations, thoughts and actions, treatments administered, patient history, medication and allergy lists, vaccine administration lists, laboratory reports, X-rays, charts, progress notes, consultation reports, procedure notes, hospital reports, correspondence, and test results) and so forth.

The enrollment and retention module 204 may be designed using patient centricity. The enrollment and retention module 204 may be configured to enable the patients/research participants to input their information online and to determine which clinical trial they would like to participate in. The enrollment and retention module 204 may be configured to enable the patients/research participants to access their medical charts (mychart) to review and update their medical history. This will enable more accurate and timely research data collection.

The list generating module 206 may be configured to generate a list of qualified subjects (list of research participants) based on the inclusion/exclusion criteria and the research participant information. The list generating module 206 may be configured to identify patients' data stored in the central database 108 and generate the qualified list of research participants to participate in the study. This may reduce a lot of intense manual labour in reviewing patient charts for study protocols. Based on built-in algorithms the system is able to identify patients that may qualify for the clinical trial.

The text remainder generating module 208 may be configured to send a notification/text message to the group/list of qualified research participants on the research participant computing device 102a to seek interest in the research study. Further, the text remainder generating module 208 may also be configured to send the remainder text to the research participant computing device 102a from the research site manager computing device 102b over the network 104 based on the scheduled appointment. The qualified group of research participants may visit a site location for the research study screening appointment. The principal investigator may perform the screening test on the qualified group of research participants for the research study at the research site location.

The appointment scheduling module 210 may be configured to enable the group of qualified research participants to request the research site manager for a research study screening appointment, and/or for adding the additional information or to schedule an online appointment. The research site manager computing device 102b may be configured to receive the request from the research participant computing device 102a to schedule the appointment for the group of qualified research participants. The appointment scheduling module 210 may be configured to enable the patients to opt-in to receive automated text reminders once a patient is qualified for a study and scheduled for an appointment for the entire duration of the study participation.

The research study documents are currently stored at the research site in three-ring binders and manual sifted through and scanned to the sponsor. The E-regulatory module 212 may allow electronic storage of records which can be easily accessed by the sponsor and may eliminate printing large volumes of paper and manual hours at the research site. The E-regulatory module 212 may be configured to enable sites to get approval faster while improving drug delivery timelines while optimizing the process of collection of the regulatory documents from the sites and eliminating errors and 483's. The regulatory documents may include, but not limited to, the financial disclosure forms, study start-up documents and checklist, training materials and documents, delegation log, curriculum vitae, temperature logs, institutional review board communication, sponsor communication, investigational product accountability records, machine learning and the like.

The primary source data capturing module 214 may be configured to capture the screening data of the group of research participants on the research site manager computing device 102b. The primary source data capturing module 214 may be an electronic data capture module. The screening data may include, but not limited to, labs and study visit procedures of the qualified group of research participants, and so forth. The study visit procedures may be varied and may require different procedures to perform which are highly customized depending on the study-specific needs. The primary source data capturing module 214 may be configured to collect research participant information in real-time to improve the quality and speed.

The primary source data capturing module 214 may be configured to collect patient data for each study protocol at the physician's office analysis knowledge answers research site. This patient's data can be collected in customized forms real-time electronically. The forms are built-in with protocol-specific validation to avoid human error during data collection. This process may help sites to adhere to the ALCOA (Attributable, Legible, Contemporaneous, Original, and Accurate) principles and improve speed and quality. The primary source data capturing module 214 may be configured to provide access to the research site manager to upload the screening reports which can be sent to each specific individual (each qualified research participant) for electronic signatures and are submitted to the sponsor for review thereby sending to an institutional review board (IRB) for approval.

The data review and reports generating module 216 may be configured to enable the sponsor to review the screening data on the sponsor computing device 102d which is captured in real-time at the research site location. The data review and reports generating module 216 may be configured to enable the research site manager to review the screening data and generates the screening reports as the amount of money that is owed to the research site location. The data review and reports generating module 216 may be configured to enable the sponsors to review the screening data for each subject/patient visit remotely and issue questions and tag study coordinators in real-time.

The financial module 218 may be configured to enable the research site manager and physicians to know in real-time what is outstanding for each study protocol or by site or by programmable interface eliminating manual calculation using complex excel spreadsheets. Based on the visits performed and agreed upon budget. The reports generated can be used to help collect outstanding balances from sponsors or CROs. The financial module 218 may be configured to update the screening data collected in real-time to calculate the accounts receivable from the sponsor.

Every study protocol has many invoiceable items that may pass through. Once a study is created in the system, the research sites can identify items which are pass-through or invoiceable. The invoice generating module 220 may generate invoices to be sent to third party vendors and sponsors. This process of automation of pass-through expenses may help sites collect funds which are usually hidden or forgotten at the end of the study. The invoice generating module 220 may be configured to generate an invoice simultaneously and periodically based on the screening reports and send the invoice to the sponsor computing device 102d over the network 104.

In accordance with one or more exemplary embodiments of the present disclosure, the metrics updating module 222 may be configured to enable the research site manager and the sponsor to update the principal investigator metrics and the site metrics (research site manager metrics) on the research site manager computing device 102b and the sponsor computing device 102d in real-time. The metrics updating module 222 may be configured to provide detailed metrics for the research site manager and the sponsor to exactly represent how many studies were completed by the physicians, in which therapeutic area, with real-time enrollment numbers, quality, and audit information. The metrics updating module 222 may be configured to store research site capabilities with pictures and the research participant testimonials made available for the sponsor. The metrics updating module 222 is a site dashboard which may allow sponsors to determine site capabilities, currently enrolling studies, list of unidentified subject lists that may qualify for a study.

Patient retention is important during the course of the conduct of the study. The E-consent/subject retention module 224 may be configured to provide tools that are simple and easily accessible by patients and may help in compliance and data collection. The E-consent/subject retention module 224 may be configured to optimize the process of electronic signature, documentation, storage and reporting.

The user feedback module 226 may be configured to enable the research site manager to provide CRA reviews to the sponsor in real-time. The user feedback module 226 may be configured to enable the research site manager to provide feedback to the sponsor. The user feedback module 226 may also be configured to enable the sponsor to provide feedback to the research site manager. During the course of the study collecting adverse events and information from patients regarding their health in real-time is critical to the success and compliance of the study protocol. The user feedback module 226 may be configured to optimize the data that needs to be collected from patients more accurate and timelier. The user feedback module 226 may be configured to increase patient retention and optimize the process of communication between the patient and site staff.

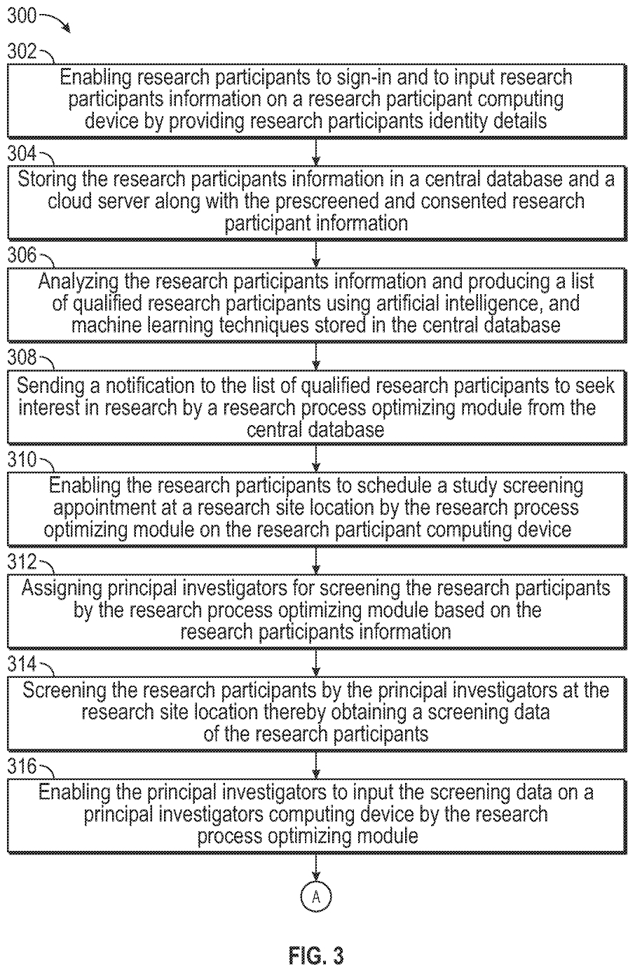

Referring to FIG. 3 is a flow diagram 300 depicting a method for providing an optimized process in clinical research, in accordance with one or more exemplary embodiments. The method 300 may be carried out in the context of the details of FIG. 1, and FIG. 2. However, the method 300 may also be carried out in any desired environment. Further, the aforementioned definitions may equally apply to the description below.

The method commences at step 302, enabling research participants to sign-in and to input research participants information on a research participant computing device by providing research participants' identity details. Thereafter at step 304, storing the research participants' information in the central database and the cloud server along with the pre-screened and consented research participant information. Thereafter at step 306, analyzing the research participants' information and sorting/producing a list of qualified research participants using artificial intelligence, and machine learning techniques stored in the central database. Thereafter at step 308, sending a notification to the list of qualified research participants to seek study in research by the research process optimizing module. Thereafter at step 310, enabling the research participants to schedule a study screening appointment at a research site location by the research process optimizing module on the research participant computing device. Thereafter at step 312, assigning principal investigators for screening the research participants by the research process optimizing module based on the research participants information. Thereafter at step 314, screening the research participants by the principal investigators at the research site location thereby obtaining a screening data of the research participants. Thereafter at step 316, enabling the principal investigators to input the screening data on a principal investigators computing device by the research process optimizing module. Thereafter at step 318, enabling the research site manager to review the screening data on the research site manager computing device by the research process optimizing module. Thereafter at step 320, generating screening reports as the amount of money that is owed to the research site location by the research process optimizing module. Thereafter at step 322, generating an invoice by the research process optimizing module based on the screening reports. Thereafter at step 324, reporting the invoice to a sponsor by the research process optimizing module and enabling the sponsor to review the invoice on a sponsor computing device.

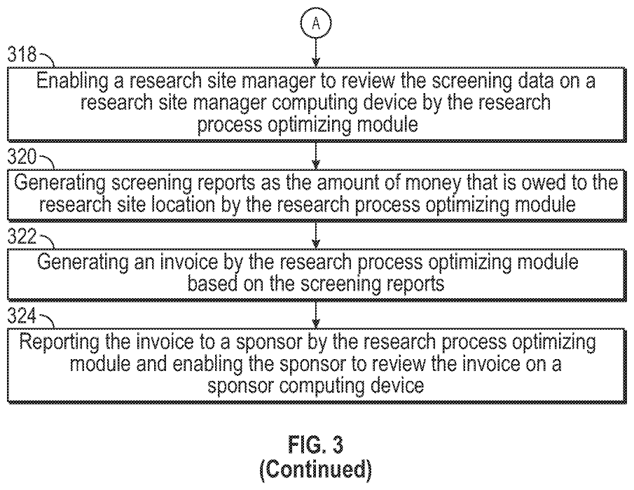

Referring to FIG. 4 is another exemplary flow diagram 400 depicting a method of an optimized process that improves efficiency and quality in clinical research, in accordance with one or more exemplary embodiments. The method 400 may be carried out in the context of the details of FIG. 1, FIG. 2, and FIG. 3. However, the method 400 may also be carried out in any desired environment. Further, the aforementioned definitions may equally apply to the description below.

The method commence at step 402, enabling patients/research site staff to sign-in and to input patient information on the research participant computing device/research site manager computing device by the user registration module. Thereafter at step 404, sending the patient information to the central database and the cloud server over the network. Thereafter at step 406, storing the patients' information in the central database and the cloud server along with the pre-screened and consented patients' information. Thereafter at step 408, generating/sorting a list of qualified subjects by the qualified list generating module based on inclusion/exclusion criteria. Thereafter at step 410, sending a text notification to the list of qualified subjects by the remainder generating module to seek interest in the research study. Thereafter at step 412, allowing the subjects to call research sites for adding additional information or to schedule an appointment by an appointment scheduling module. Thereafter at step 414, sending a reminder text to the subjects regarding the appointment by the remainder generating module. Thereafter at step 416, screening the subjects for the research study by the principal investigators. Thereafter at step 418, enabling the principal investigators to enter the subject labs and study visit procedures by the primary source data capture module. Thereafter at step 420, enabling the principal investigators to sign off in real-time while performing history and physical examination. Thereafter at step 422 updating the total accounts receivable from the sponsor by the financial module. Thereafter at step 424, generating the screening reports as the amount of money that is owed to the research site location by the data review and reports generating module. Thereafter at step 426, generating an invoice by the invoice generating module based on the screening reports. Thereafter at step 428, reporting the invoice to the sponsor by the invoice generating module.

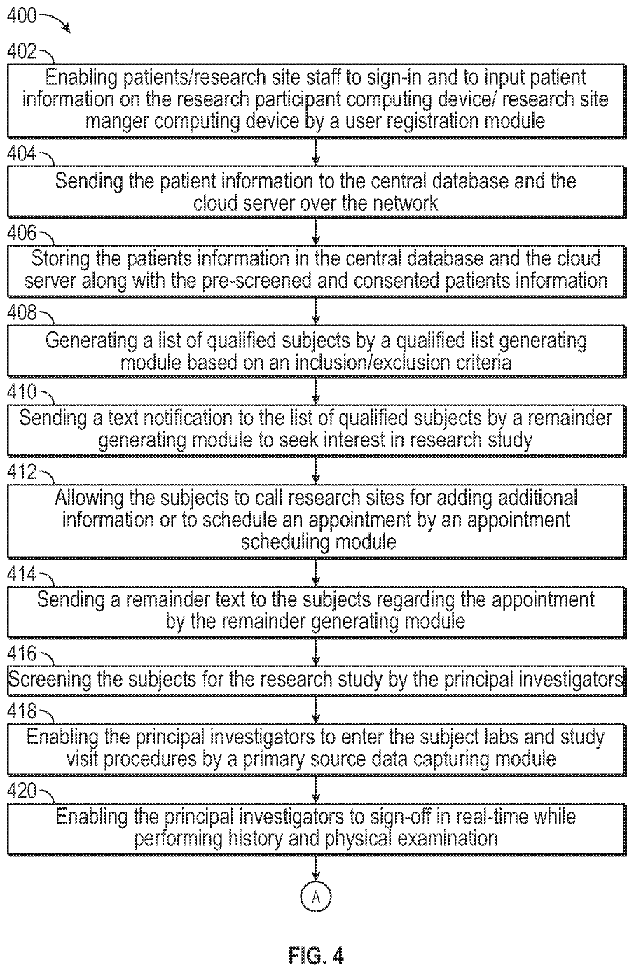

Referring to FIG. 5 is a flow diagram 500 depicting a method of an optimized process that improves efficiency and quality in clinical research, in accordance with one or more exemplary embodiments. The method 500 may be carried out in the context of the details of FIG. 1, FIG. 2, FIG. 3 and FIG. 4. However, the method 500 may also be carried out in any desired environment. Further, the aforementioned definitions may equally apply to the description below.

The method commences at step 502, patients sign-up online and enter patient information or by research site staff. Thereafter at step 504, the patient information includes diagnosis and medical history. Thereafter at step 506, the central database contains the subjects' medical history and diagnosis. Thereafter at step 508, a list of qualified subjects is generated based on the inclusion/exclusion criteria. Thereafter at step 510, sending a text to all qualified subjects to seek interest in study. Thereafter at step 512, allowing subjects to call sites for additional information or appointment, online appointments. Thereafter at step 514, the subject is scheduled for an appointment. Thereafter at step 516, a reminder text is sent to the subject regarding the appointment. Thereafter at step 518, the subject comes to the site for the study screening appointment. Thereafter at step 520, the subject is screened for the research study. Thereafter at step 522, subject labs and study visit procedures are entered in the primary source data capture module. Thereafter at step 524, the principal investigator can sign off in real-time while performing a history and physical examination. Thereafter at step 526, the subject visit is completed. Thereafter at step 528, financial information is updated for total accounts receivable from the sponsor by the financial module. Thereafter at step 530, the financial module is updated. Thereafter at step 532, a report or invoice is sent to the sponsor.

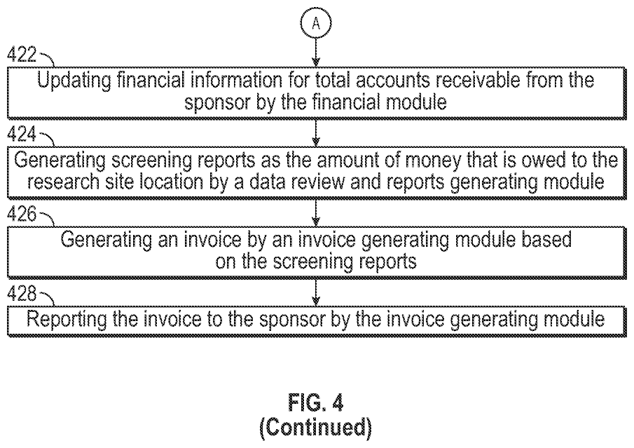

Referring to FIG. 6 is a flow diagram 600 depicting a method for sorting the research participants information into a central database and selecting the suitable research participants thereby contacting the suitable research participants for the relevant trial in clinical research, in accordance with one or more exemplary embodiments. The method 600 may be carried out in the context of the details of FIG. 1, FIG. 2, FIG. 3, FIG. 4 and FIG. 5. However, the method 600 may also be carried out in any desired environment. Further, the aforementioned definitions may equally apply to the description below.

The method commence at step 602, enabling the research participants/research site staff to sign in and to input the research participants' information on the research participant computing device/research site manage computing device by the research process optimizing module. Thereafter at step 604, the research participants' information includes the diagnosis and the medical history. Thereafter at step 606, storing the research participants' information in the central database. Thereafter at step 608, generating the list of qualified research participants by the central database based on the inclusion/exclusion criteria. Thereafter at step 610, sending the text to the list of qualified research participants by the research process optimizing module to seek interest in the research study. Thereafter at step 612, enabling the research participants to request the research sites for additional information by the research process optimizing module.

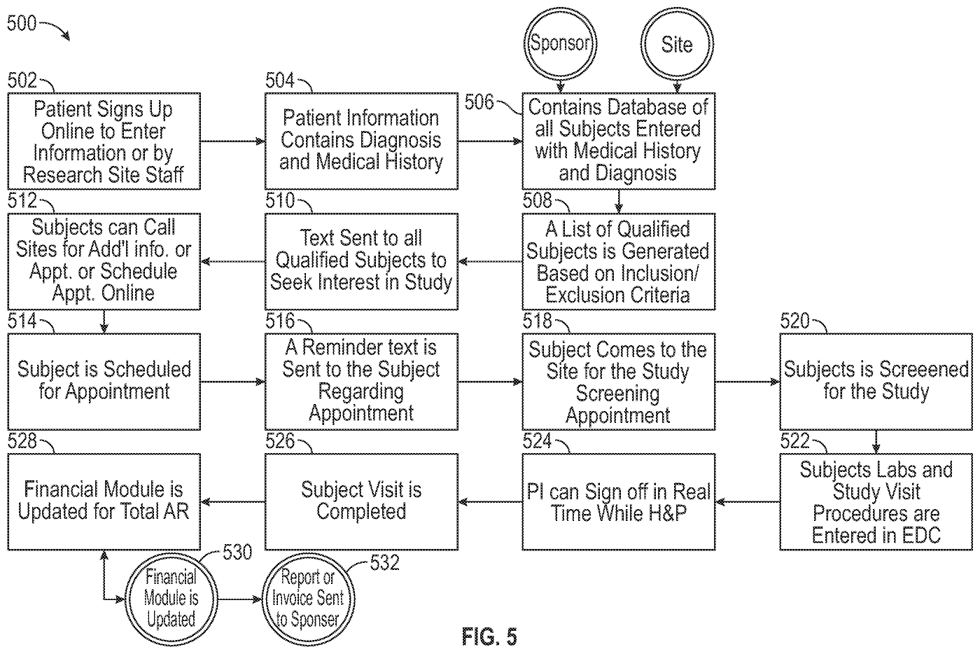

Referring to FIG. 7 is a flow diagram 700 depicting a method for scheduling an appointment and screening the research participants and entering the relevant research participants information into the primary source data capture thereby generating the financial information and invoices automatically, in accordance with one or more exemplary embodiments. The method 700 may be carried out in the context of the details of FIG. 1, FIG. 2, FIG. 3, FIG. 4, FIG. 5, and FIG. 6. However, the method 700 may also be carried out in any desired environment. Further, the aforementioned definitions may equally apply to the description below.

The method commence at step 702, enabling the research participants' to schedule the screening appointment at research sites by the research process optimizing module. Thereafter at step 704, sending the reminder text to the research participants' regarding the appointment by the research process optimizing module. Thereafter at step 706, attending the research participants' to the research site location for the study screening appointment. Thereafter at step 708, screening the research participants' for the research study. Thereafter at step 710, enabling the research staff/principal investigator to enter the research participants' labs and the study visit procedures by the primary source data capture module. Thereafter at step 712, enabling the principal investigator to sign off in real-time while performing the history and physical examination. Thereafter at step 714, completing the research participants' visit. Thereafter at step 716, updating the financial information for the total accounts receivable from the sponsor by the financial module. Thereafter at step 718, reporting or sending the invoice to the sponsor by the research process optimizing module.

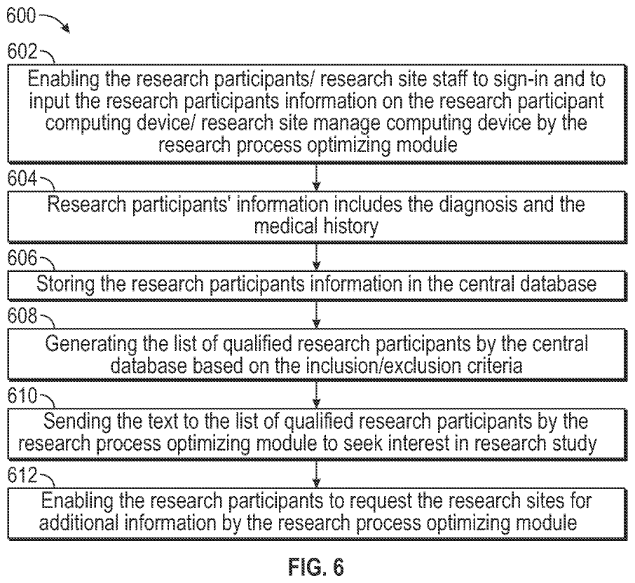

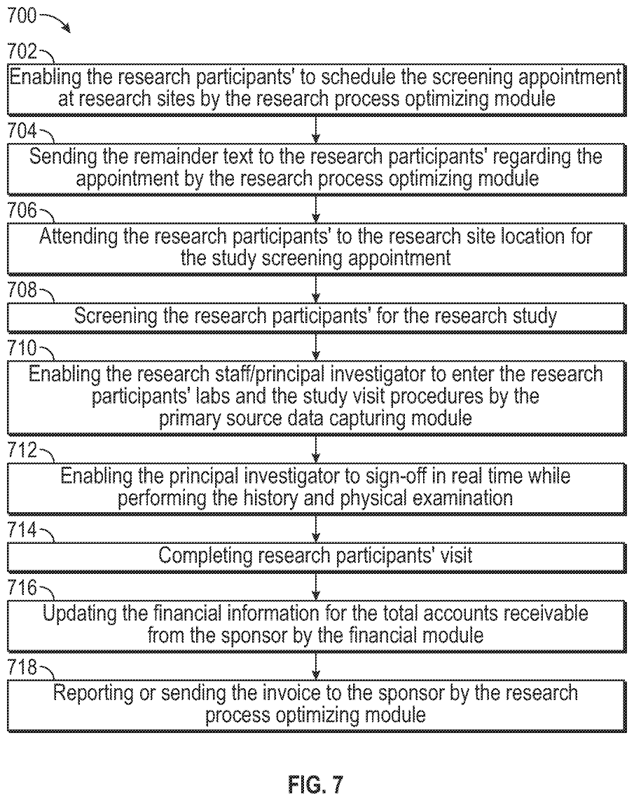

Referring to FIG. 8A is a flow diagram 800a depicting a method for uploading documents by the e-regulatory module, in accordance with one or more exemplary embodiments. The method 800a may be carried out in the context of the details of FIG. 1, FIG. 2, FIG. 3, FIG. 4, FIG. 5, FIG. 6 and FIG. 7. However, the method 800a may also be carried out in any desired environment. Further, the aforementioned definitions may equally apply to the description below.

The method commence at step 802, enabling the research site manager to sign in on the research site manager computing device by the research process optimizing module. Thereafter at step 804, enabling the research site manager to upload the documents by the e-regulatory module. Thereafter at step 806, providing access to the sponsor or chief medical officer (CMO) for download.

Referring to FIG. 8B is a flow diagram 800b depicting a method for sharing the research participant information between the research participant, the research site, and the sponsor, in accordance with one or more exemplary embodiments. The method 800b may be carried out in the context of the details of FIG. 1, FIG. 2, FIG. 3, FIG. 4, FIG. 5, FIG. 6, FIG. 7 and FIG. 8A. However, the method 800b may also be carried out in any desired environment. Further, the aforementioned definitions may equally apply to the description below.

The method commence at step 808, enabling the principal investigator and the research site manager to sign in on the principal investigator computing device and the research site manager computing device by the research process optimizing module. Thereafter at step 810, enabling the principal investigator to update the principal investigator metrics in real-time by the research process optimizing module. Thereafter at step 812, enabling the research site manager to update the research site metrics in real-time by the research process optimizing module. Thereafter at step 814, enabling the sponsors to review the principal investigator and site metrics on the sponsor's computing device. Thereafter at step 816, enabling the research site manager to review the principal investigator and site metrics on the research site manager computing device. Thereafter at step 818, enabling the research site manager to provide feedback to the sponsors through the research process optimizing module. Thereafter at step 820, enabling the sponsors to provide feedback by the research process optimizing module on the sponsor computing device.

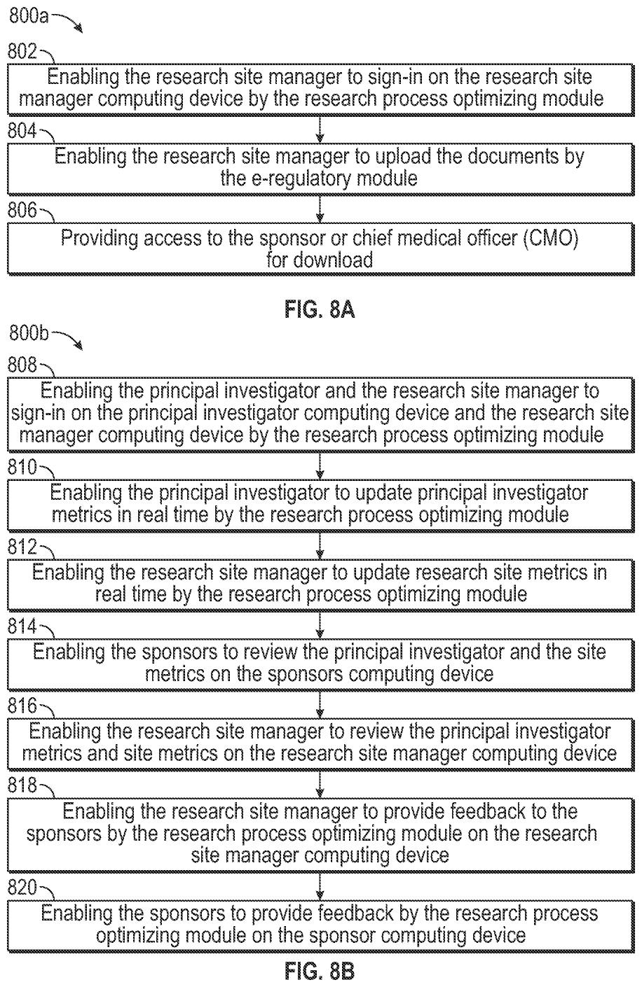

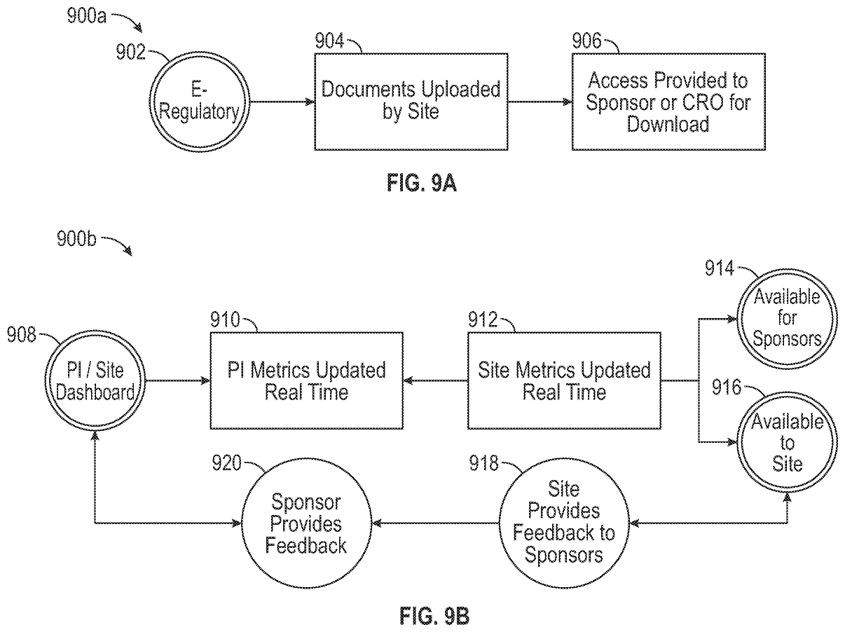

Referring to FIG. 9A is another exemplary flow diagram 900a depicting the method for uploading documents by the e-regulatory module, in accordance with one or more exemplary embodiments. The method 900a may be carried out in the context of the details of FIG. 1, FIG. 2, FIG. 3, FIG. 4, FIG. 5, FIG. 6, FIG. 7, FIG. 8A, and FIG. 8B. However, the method 900a may also be carried out in any desired environment. Further, the aforementioned definitions may equally apply to the description below.

The method commence at step 902, enabling the research site manager to access the e-regulatory module. Thereafter at step 904, uploading the documents by the research site manager using the e-regulatory module. Thereafter at step 906, providing access to the sponsor or chief medical officer (CMO) for download.

Referring to FIG. 9B is another exemplary flow diagram 900b depicting the method for sharing the research participant information between the research participant, the research site, and the sponsor, in accordance with one or more exemplary embodiments. The method 900b may be carried out in the context of the details of FIG. 1, FIG. 2, FIG. 3, FIG. 4, FIG. 5, FIG. 6, FIG. 7, FIG. 8A, FIG. 8B, and FIG. 9A. However, the method 900b may also be carried out in any desired environment. Further, the aforementioned definitions may equally apply to the description below.

The method commence at step 908, enabling the principal investigator and the research site manager to access the PI/site dashboard. Thereafter at step 910, updating the principal investigator metrics in real-time. Thereafter at step 912, updating the research site metrics in real-time. Thereafter at step 914, enabling the sponsors to review the principal investigator metrics and site metrics. Thereafter at step 916, enabling the research site manager to review the principal investigator metrics and site metrics. Thereafter at step 918, enabling the research site manager to provide feedback to the sponsors. Thereafter at step 920, enabling the sponsors to provide feedback.

Referring to FIG. 10 is a block diagram 1000 illustrating the details of a digital processing system 1000 in which various aspects of the present disclosure are operative by execution of appropriate software instructions. The Digital processing system 1000 may correspond to the research participant computing device 102a research site manager computing device 102b, or the principal investigator computing device 102c, and the sponsor computing device 102d (or any other system in which the various features disclosed above can be implemented).

Digital processing system 1000 may contain one or more processors such as a central processing unit (CPU) 1010, random access memory (RAM) 1020, secondary memory 1030, graphics controller 1060, display unit 1070, network interface 1080, and input interface 1090. All the components except display unit 1070 may communicate with each other over communication path 1050, which may contain several buses as is well known in the relevant arts. The components of FIG. 10 are described below in further detail.

CPU 1010 may execute instructions stored in RAM 1020 to provide several features of the present disclosure. CPU 1010 may contain multiple processing units, with each processing unit potentially being designed for a specific task. Alternatively, CPU 1010 may contain only a single general-purpose processing unit.

RAM 1020 may receive instructions from secondary memory 1030 using communication path 1050. RAM 1020 is shown currently containing software instructions, such as those used in threads and stacks, constituting shared environment 1025 and/or user programs 1026. Shared environment 1025 includes operating systems, device drivers, virtual machines, etc., which provide a (common) run time environment for execution of user programs 1026.

Graphics controller 1060 generates display signals (e.g., in RGB format) to display unit 1070 based on data/instructions received from CPU 1010. Display unit 1070 contains a display screen to display the images defined by the display signals. Input interface 1090 may correspond to a keyboard and a pointing device (e.g., touch-pad, mouse) and may be used to provide inputs. Network interface 1080 provides connectivity to a network (e.g., using Internet Protocol), and may be used to communicate with other systems (such as those shown in FIG. 1) connected to the network 104.

Secondary memory 1030 may contain hard drive 1035, flash memory 1036, and removable storage drive 1037. Secondary memory 1030 may store the data software instructions (e.g., for performing the actions noted above with respect to the Figures), which enable digital processing system 1000 to provide several features in accordance with the present disclosure.

Some or all of the data and instructions may be provided on removable storage unit 1040, and the data and instructions may be read and provided by removable storage drive 1037 to CPU 1010. Floppy drive, magnetic tape drive, CD-ROM drive, DVD Drive, Flash memory, removable memory chip (PCMCIA Card, EEPROM) are examples of such removable storage drive 1037.

Removable storage unit 1040 may be implemented using medium and storage format compatible with removable storage drive 1037 such that removable storage drive 1037 can read the data and instructions. Thus, removable storage unit 1040 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 1040 or hard disk installed in hard drive 1035. These computer program products are means for providing software to digital processing system 1000. CPU 1010 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 1030. Volatile media includes dynamic memory, such as RAM 1020. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 1050. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A system for providing an optimized process in a clinical research, comprising:
a research process optimizing module embedded in a memory of a research participant's computing device, a research site manager's computing device, and a principal investigator's computing device, and a sponsor's computing device, whereby the research process optimizing module configured to collect, sort and process one or more research participants' information to provide the optimized process in the clinical research, the research process optimizing module comprises:
an enrolment and retention module configured to enable at least of: a research participant; and a site manager to input the one or more research participants' information on at least one of: the research participant's computing device; and the research site manager computing device over a network;
a list generating module configured to analyse the one or more research participants' information stored in the central database and generate a list of qualified research participants to participate in the clinical research based on at least one of: an exclusion criteria; and an inclusion criteria; and the one or more research participants' information using artificial intelligence, and machine learning techniques;
an appointment scheduling module configured to enable the list of qualified research participants to input additional patients information on the research participant's computing device and to schedule an appointment with the site manager for research study screening, whereby the appointment scheduling module configured to enable the one or more research participants to send a request from the research participant's computing device to the research site manager's computing device for scheduling the appointment, the appointment scheduling module configured to assign one or more principal investigators for screening one or more research participants based on the one or more research participants' information;
a primary source data capturing module configured to enable one or more principal investigators to input screening data of the one or more research participants on the principal investigator's computing device after screening the one or more research participants at a research site location in real-time;
a financial module configured to collect the screening data in real-time and calculate accounts receivable from a sponsor thereby send the screening data to a data review and reports generating module, wherein the data review and reports generating module configured to generate one or more screening reports as the amount of money that is owed to a research site location and send the one or more screening reports to an invoice generating module; and
an invoice generating module configured to generate an invoice based on the one or more screening reports of the one or more research participants and send the invoice to the sponsor's computing device, the data review and reports generating module configured to enable the sponsor to review the invoice on the sponsor computing device.

2. The system of claim 1, wherein the one or more research participants' information comprises health history, medical reports, and diagnosis reports.

3. The system of claim 1, wherein the enrolment and retention module is configured to send the one or more research participants' information to a cloud server and a central database over the network.

4. The system of claim 3, wherein the cloud server and the central database is configured to store the one or more research participants' information.

5. The system of claim 1, wherein the one or more research participants' information comprises diagnosis and medical history, patients' demographic information (e.g., age, weight, gender, race, income, and geographic location), and health-related information (e.g., clinician documentation of observations, thoughts and actions, treatments administered, patient history, medication and allergy lists, vaccine administration lists, laboratory reports, X-rays, charts, progress notes, consultation reports, procedure notes, hospital reports, correspondence, and test results).

6. The system of claim 1, wherein the research process optimizing module comprises a user registration module configured to enable at least one of: the research participants; the principal investigator; and the sponsor to register on at least one of: the research participant computing device; the principal investigator computing device; and the sponsor computing device by providing at least one of: user identity credentials; principal investigator identity credentials; and sponsor identity credentials.

7. The system of claim 1, wherein the enrolment and retention module is configured to optimize and improve the experience and engagement of the one or more research participants in the clinical research.

8. The system of claim 1, wherein the research process optimizing module comprises a text remainder generating module configured to send one or more notifications to the research participant's computing device to seek interest in a research study.

9. The system of claim 8, wherein the text remainder generating module is configured to send a reminder text to the research participant computing device from the research site manager computing device over the network.

10. The system of claim 1, wherein the appointment scheduling module is configured to enable the one or more research participants to opt-in to receive automated text reminders once a research participant is qualified for the clinical research study and scheduled for an appointment for their entire duration of the study participation.

11. The system of claim 1, wherein the research process optimizing module comprises an E-regulatory module configured to enable the sponsor to access electronic storage of records easily and eliminate printing large volumes of paper and manual hours at the research site location.

12. The system of claim 1, wherein the primary source data capturing module is configured to capture the screening data of the list of research participants on the research site manager computing device in real-time.

13. The system of claim 1, wherein the primary source data capturing module is configured to provide access to the research site manager to upload the one or more screening reports which can be sent to each qualified research participant for electronic signatures and are submitted to the sponsor for review thereby sending to an institutional review board (IRB) for approval.

14. The system of claim 1, wherein the data review and reports generating module is configured to enable the site manager to review the screening data of the one or more research participants on the research site manager computing device.

15. The system of claim 1, wherein the research process optimizing module comprises a metrics updating module configured to enable at least one of: the research site manager; and the sponsor to update at least one of: the principal investigator metrics; and the site metrics on at least one of: the research site manager computing device; and the sponsor computing device in real-time.

16. The system of claim 1, wherein the research process optimizing module comprises an E-consent/subject retention module configured to provide one or more tools that are simple and easily accessible by the one or more research participants and help in compliance and data collection.

17. A method for providing an optimized process in clinical research, comprising;
enabling at least of: a research participant; and a site manager to input one or more research participants' information by an enrolment and retention module on at least one of: a research participant computing device; and a research site manager computing device over a network;
sending the one or more research participants' information from the enrolment and retention module to a cloud server and a central database over the network;
identifying the one or more research participants' information stored in the central database and generating a list of qualified research participants to participate in the clinical research by a list generating module;
enabling the list of qualified research participants to input additional patients information by the appointment scheduling module on the research participant computing device;
enabling the list of qualified research participants to schedule an appointment with the site manager for research study screening by the appointment scheduling module;
enabling the list of qualified research participants to send a request from the research participant's computing device to the research site manager's computing device for scheduling the appointment;
assigning one or more principal investigators to screen the one or more research participants by the appointment scheduling module based on the one or more research participants' information;
enabling one or more principal investigators to input screening data of the one or more research participants by a primary source data capturing module on the principal investigators computing device after screening the one or more research participants at a research site location in real-time;
collecting the screening data in real-time and calculating accounts receivable from a sponsor by a financial module thereby sending the screening data to a data review and reports generating module;
generating one or more screening reports by the data review and reports generating module as the amount of money that is owed to the research site location;
sending the one or more screening reports to the invoice generating module and generating an invoice based on the one or more screening reports of the one or more research participants; and
sending the invoice to the sponsor computing device and enabling the sponsor to review the invoice generated based on the one or more screening reports of the one or more research participants.

18. The method of claim 16, comprising a step of generating the list of qualified research participants based on at least one of: exclusion criteria; inclusion criteria; and the one or more research participants' information using artificial intelligence and machine learning techniques.

19. A computer program product comprising a non-transitory computer-readable medium having a computer-readable program code embodied therein to be executed by one or more processors, said program code including instructions to:
enable at least of: a research participant; and a site manager to input one or more research participants' information by enrolment and retention module on at least one of: a research participant computing device; and a research site manager computing device over a network;
send the one or more research participants' information from the at least one of: the research participant computing device; and the research site manager computing device to a cloud server and a central database by the enrolment and retention module over the network;
analyze the one or more research participants' information stored in the central database and generate a list of qualified research participants to participate in the clinical research by a list generating module;
enable the list of qualified research participants to input additional patients information by the appointment scheduling module on the research participant computing device;
enable the list of qualified research participants to schedule an appointment with the site manager for research study screening by the appointment scheduling module;
enabling the list of qualified research participants to send a request from the research participant's computing device to the research site manager's computing device for scheduling the appointment;
assign one or more principal investigators to screen the one or more research participants by the appointment scheduling module based on the one or more research participants' information;
enable one or more principal investigators to input screening data of the one or more research participants by a primary source data capturing module on the principal investigators computing device after screening the one or more research participants at a research site location in real-time;
collect the screening data in real-time and calculate accounts receivable from a sponsor by a financial module thereby send the screening data to a data review and reports generating module;
generate one or more screening reports by the data review and reports generating module as the amount of money that is owed to the research site location;
send the one or more screening reports to the invoice generating module and generate an invoice based on the one or more screening reports of the one or more research participants; and
send the invoice to the sponsor computing device and enable the sponsor to review the invoice generated based on the one or more screening reports of the one or more research participants.
